(19) United States
(12) Patent Application Publication
(10) Pub. No.: US 2020/0009074 A1
(43) Pub. Date: Jan. 9, 2020

(54) SUPPLEMENT PATCH

(71) Applicant: COSMED PHARMACEUTICAL CO., LTD., Kyoto-city (JP)

(72) Inventors: Hiroshi Tanaka, Kyoto-city (JP); Ying-shu Quan, Kyoto-city (JP); Junya Hasegawa, Kyoto-city (JP); Fumio Kamiyama, Kyoto-city (JP)

(21) Appl. No.: 16/458,086

(22) Filed: Jun. 30, 2019

(30) Foreign Application Priority Data
Jul. 3, 2018 (JP) 2018-127164

(51) Int. Cl. A61K 9/70

(52) U.S. Cl. CPC A61K 9/7061

(57) ABSTRACT

[Problem]

To provide a supplement in a new form by which the targeted component is made available for intake other than the oral intake.

[Solution]

A patch-type supplement composed of a support, a component-containing layer, and a release liner, and the component-containing layer containing a supplement component and an adhesive. The adhesive is preferably an acrylate-based adhesive. A water vapor permeability of the support is preferably not larger than 1000 g/m²×24 hours. The supplement component is preferably made of an aqueous component and an organic component.

### TECHNICAL FIELD

The present invention relates to a patch-type supplement sheet.

### BACKGROUND ART

Supplements are also called nutritional supplementary food or health supplementary food and are foods having a purpose of supplementing nutrient intake such as vitamins, minerals, and acids or medical effect by components such as herbs. In Japan, there is no legal or administrative definition, but the Ministry of Health, Labour and Welfare defines the supplement for convenience as products in the form of tablets or capsules in which specific components are concentrated (Non-Patent Document 1).

Use of the health foods and supplements has been expanding for the purposes of maintenance of health, supplement of nutrient components, prevention of diseases and the like (Non-Patent Document 1).

### PRIOR ART DOCUMENTS

### Non-Patent Document

Non-Patent Document 1: http://www.mhlw.go.jp/topics/bukyoku/iyaku/syoku-anzen/dl/pamph_healthfood_d.pdf

### SUMMARY OF INVENTION

### Technical Problem

Supplements are orally taken in principle, but oral intake in the forms of tablets or capsules is difficult in some cases, and there is also a demand for keeping blood concentration of a component of the supplement constant for a long time. An object of the present invention is to provide a supplement in a new form by which the targeted component is made available for intake other than the oral intake.

### Solution to Problem

In order to solve the aforementioned problem, the inventors succeeded in development of a supplement of a type not oral but transdermal absorption by containing a supplement component in an adhesive layer of a patch and has completed the present invention as the result of keen study.

The present invention is as described below:
[1] A patch-type supplement composed of a support, a component-containing layer, and a release liner, and the component-containing layer containing a supplement component and an adhesive.
[2] The patch-type supplement according to [1], in which a water vapor permeability of the support is not larger than 1000 g/m²×24 hours.
[3] The patch-type supplement according to [1] or [2], in which the supplement component is made of an aqueous component and an organic component.
[4] The patch-type supplement according to any one of [1] to [3], in which the supplement component is made of the aqueous component and the organic component, and two kinds or more of each of the components are contained.
[5] The patch-type supplement according to any one of [1] to [4], in which at least one kind of the supplement component exists in the component-containing layer in a dissolved state, and the other at least one kind exists in the component-containing layer in a dispersed state.
[6] The patch-type supplement according to any one of [1] to [5], in which at least one kind of the supplement component is dispersed in the component-containing layer in a powder state.
[7] The patch-type supplement according to any one of [1] to [6], which is a matrix-type supplement sheet.
[8] The patch-type supplement according to any one of claims 1 to 7, in which the component-containing layer further contains at least one kind of a dissolving agent or a surfactant.
[9] The patch-type supplement according to any one of [1] to [8], in which the total of the supplement components is 5 mass parts or more to 100 mass parts of the component-containing layer.
[10] The patch-type supplement according to [9], in which the total of the aqueous component or the total of the organic component of the supplement component is 1 mass, part or more to the 100 mass parts of the component-containing layer.
[11] The patch-type supplement according to any one of [1] to [10], in which the adhesive is an acrylate-based, styrene-isoprene-styrene (SIS)-based, polyisobutylene (PIB)-based, or silicone-based adhesive.
[12] The patch-type supplement according to any one of [1] to [11], in which the adhesive is an acrylate-based adhesive, and the adhesive is crosslinked.
[13] The patch-type supplement according to any one of [1] to [12], in which the adhesive is a hydrophilic acrylate-based adhesive.
[14] The patch-type supplement according to any one of [1] to [13], in which the component-containing layer is a lamination of the supplement component-containing layer and a supplement component-free layer.

### Effects of Invention

The patch-type supplement of the present invention can keep the blood concentration of the component constant for a long time as compared with oral intake by continuous absorption of the supplement component through the skin. Since the oral intake is not required, even if swallowing of a tablet or a capsule is difficult, active components can be easily absorbed into a body. In the patch-type supplement of the present invention, since the supplement component does not go through the mouth, additives for reducing bitterness or the like deriving from the component or devising of a dosage form is not needed, and a burden is not applied to internal organs such as digestive organs.

### DESCRIPTION OF EMBODIMENTS

In the present invention, the patch-type supplement refers to a supplement in s patch format containing an active component to be orally taken as a health food or the like in an adhesive layer and used for the purpose of absorption of the active component not orally but transdermally. In the present invention, the active component contained in the patch-type supplement is referred to as a supplement component.

The patch-type supplement of the present invention is composed of a support, a component-containing layer, and a release liner. As a mode of a tape preparation which is a medicine, those composed of a support, a paste (containing an adhesive and a drug), and a release liner is called a matrix-type formulation. The matrix-type formulation as a supplement formulation is not known, and the present invention provides a novel matrix-type supplement sheet as a patch-type supplement.

The patch-type supplement of the present invention contains a supplement component and an adhesive in the component-containing layer. When seen from another aspect, a structure of the component-containing layer may be composed of an adhesive layer containing the supplement component.

The supplement component is not particularly limited as long as it is a component contained in an oral supplement or a health food. It includes, for example, vitamins, minerals, amino acids, essential fatty acids, extracts of plant and animal origins (essences) and the like.

Vitamins include vitamin A, vitamin B group, vitamin C, vitamin D, vitamin E, vitamin H, vitamin K, vitamin P, vitamin U, cobalamin, vitamin premix and the like.

Minerals include zinc, iron, copper, chromium, selenium, magnesium, calcium, potassium, sodium, cobalt, molybdenum, iodine, phosphorus and the like.

Examples of amino acids include essential amino acids such as tryptophan, threonine, valine, leucine, isoleucine, lysine, methionine, phenylalanine, histidine, non-essential amino acids such as arginine, asparagine, cysteine, tyrosine, serine, praline, glutamine, glycine, alanine, aspartic acid, glutamic acid, taurine, ornithine, gamma (γ)-amino butyric acid and the like.

Examples of the essential amino acids include alpha (α)-linolenic acid, eicosapentaenoic acid (EPA), docosahexaenoic acid (DHA), arachidonic acid, gamma (γ)-linolenic acid and the like. Acid amides include nicotinic acid amide.

Examples of materials of the extracts of plant and animal origins (essences) include materials usable as foods such as: bacteria; yeast; mushrooms; fruits; leaves, stems, fruits, roots, root stocks, petals, seeds of plants; fish; and eggs of birds. The extracts of plant and animal origins (essences) may be specific components obtained by further purifying the extracts. When Curcuma domestica is used as a material, for example, it may be turmeric which is an extract or may be a purified substance of curcumin contained in turmeric. Others include Panax ginseng essence, American ginseng essence, Eleutherococcus senticosus root essence, Ophiocordyceps sinensis, pycnogenol, black ginger essence and the like.

A content of the supplement component in the patch-type supplement of the present invention can be set as appropriate in accordance with the component. It may be indicated by a ratio to a mass of the entire component-containing layer or may be indicated by a content (mass) per patch-type supplement. The patch-type supplement of the present invention is characterized by a variety of component contained, whereby each component acts synergistically, and effectiveness is improved. More specifically, the supplement component is a mixture of an aqueous component and an organic component, and it is preferable that two kinds or more or preferably three kinds or more of the respective components are contained. In the present invention, the aqueous component refers to a component with water as a good solvent, and the organic component refers to a component exhibiting solubility in ethanol higher than water. In an example, the active components noted with purified water in sections of A, B, and C are aqueous components, and the active components noted with ethanol are organic components.

At least one kind of the organic components in the supplement components may exist in the component-containing layer in a dissolved state, and at least one kind of the aqueous components in the supplement components may exist in the component-containing layer in a dispersed state.

A preferred mode of the patch-type supplement of the present invention is a matrix-type supplement sheet in which a plurality of supplement components is dissolved and/or dispersed with an adhesive as a base.

As an example of the contents of the supplement components, the total of the supplement components is 5 mass parts or more and 80 mass parts or less with respect to 100 mass parts of the component-containing layer. The breakdown of the contents is that the total of the aqueous components or the total of the organic contents is 1 mass part or more and 79 mass parts or less to 100 mass parts of the component-containing layer.

The patch-type supplement of the present invention includes a silicone-based adhesive, an acrylate-based adhesive, or a rubber-based (polyisobutylene-based; polystyrene-based such as styrene-isoprene-styrene) adhesive as the base in the component-containing layer. The acrylate-based adhesive is preferable. In the acrylate-based adhesives, a hydrophilic acrylate-based adhesive is more preferable. That is because an adhesive property to the skin and the adhesive property particularly when sweating are excellent. The hydrophilic acrylate-based adhesive in the present invention is an acrylate-based adhesive containing 10 mass % or more of acrylic acid, 15 mass % or more of hydroxyethyl (meth)acrylate or 20 mass % or more of methoxyethyl acrylate in an acryl comonomer.

The patch-type supplement of the present invention can include other components other than the supplement components in the adhesive. For example, in order to mix a water-soluble component and an oil-soluble component well, solubilizing agents such as glycerin, propylene glycol (PG), 1,3-butylene glycol (BG), surfactants such as fatty acid ester, sorbitan fatty acid ester, glycerin fatty acid ester, polyoxyethylene sorbitan monooleate (Tween 80), sobitan monooleate (Span 80) and the like; in order to improve adhesiveness, plasticizers such as isopropyl myristate, octyldodecyl lactate; antioxidants such as BHT for keeping stability of the supplement components; crosslinking agents, fillers and the like for making a system robust in order to prevent remaining of a paste and the like are examples. As a preferred embodiment, the adhesive is an acrylate-based adhesive, and the adhesive is crosslinked. The crosslinking agent to be used is preferably an isocyanate crosslinking agent.

A thickness of the component-containing layer depends on a purpose of use but it is preferably 10 to 200 μm. The supplement component contained in the component-containing layer can be contained by being dissolved or dispersed in the adhesive.

At least one kind of supplement components may exist in the component-containing layer in the dissolved state, and at least another kind may exist in the component-containing layer in the dispersed state. As a preferred dispersed state, at least one kind of the supplement components may be dispersed in a powder state. The component-containing layer may be composed of the supplement component-containing layer and the supplement component-free layer and may be a lamination of the supplement component-containing layer and the supplement component-free layer. Here, the supplement component-containing layer and the supplement component-free layer are the supplement component-containing layer and the adhesive layer not containing the supplement component. In this case, the configuration of the patch-type supplement includes a support/supplement component-containing layer/supplement component-free layer/release liner, a support/supplement component-free layer/supplement component-containing layer/release liner or a support/supplement component-free layer/supplement component-containing layer/supplement component-free layer/release liner or the like. By laminating the supplement component-containing layer and the supplement component-free layer, adhesion of the supplement component-containing layer to the support or close contact property between the skin side and the patch-type supplement can be improved. When the component-containing layer is lamination, the thickness of the entire lamination only needs to be set to approximately 10 to 200 μm.

For the support in the patch-type supplement of the present invention, a stretchable or non-stretchable support usually used for transdermal absorption type formulation. Examples of the support include synthetic resin films or sheets of polyethylene, polypropylene, polybutadiene, ethylene-vinyl acetate copolymer, polyvinylchloride, polyester (such as polyethylene terephthalate (PET)), nylon, polyurethane and the like or a laminated body, a porous body, a foamed body, paper, woven cloth, unwoven cloth and the like thereof in order to assist penetration of the supplement component into the skin, a support with small water vapor permeability is preferable. If the water vapor permeability is low, penetration of valuable components into the skin is improved by an ODT (occlusive dressing technique) effect and thus, the water vapor permeability is preferably 1000 g/m²×24 hours or less and more preferably 500 g/m²×24 hours or less. The water vapor permeability is a value measured in accordance with JIS Z 0208: Testing methods for determining water vapor transmission rate of moisture-proof packaging materials (Cup method). Calcium chloride is used as a moisture adsorbent.

The support may have a two-layered or multi-layered structure of a synthetic resin film or sheet or a laminated body thereof and a porous body, a foamed body, paper, woven cloth or unwoven cloth. The two-layered structure made of polyethylene terephthalate and unwoven cloth or the two-layered structure made of polyethylene terephthalate and ethylene-vinyl acetate copolymer is preferable, for example. The thickness of the support is preferably 10 to 100 μm.

For the release liner in the patch-type supplement of the present invention, an active-component impermeable release liner is used. Examples of the release liner include a film made of a polymer material such as polyethylene, polypropylene, polyester, the one in which aluminum is deposited to a film, the one in which silicone oil or the like is applied on paper and the like. Among them, a polyester film is preferable since active components are not permeated and also from points of workability and a low cost, and polyethylene terephthalate (PET) film is particularly preferable. Moreover, examples of the release liner include a laminate film obtained by bonding a plurality of materials together. The thickness of the release liner is 200 μm or less, preferably 10 to 100 μm.

Examples of the shape of the patch-type supplement of the present invention include a circle, an oval, a rectangle, a regular square and the like and are not particularly limited. A plane area of the sheet is preferably 0.5 to 100 cm². It is more preferably 0.7 to 30 cm². Moreover, in order to make the patch-type supplement easier to be released from the release liner, a slit may be formed over a part or the whole of the release liner side. A cutout may be formed at an end of the release liner.

A manufacturing method of the patch-type supplement of the present invention is not particularly limited, and the patch-type supplement can be manufactured in accordance with a well-known manufacturing method of transdermal absorption formulation. Examples of the manufacturing method include a method in which the supplement component and the adhesive and the like are dissolved in an organic solvent such as ethyl acetate, hexane, toluene or a mixed solvent thereof, this dissolved substance is spread on the release liner or the support, the solvent in the dissolved substance is evaporated so as to form the component-containing layer and then, the support or the release liner is bonded together, whereby the patch-type supplement is obtained or a method in which the supplement component and the adhesive and the like are heated/melted, this melted substance is spread on the release liner or the support so as to form the component-containing layer and then, the support or the release liner is bonded together, whereby the patch-type supplement is obtained.

A part on which the patch-type supplement of the present invention is to be pasted is not particularly limited, but a part on the skin which is clean and dry and less hair is recommended. More specifically, the breast (a part with less motion), an upper arm and the like are preferable. The number of pasting times only needs to be once a day but it may be twice or three times a day by changing the pasting portion. The number of patches to be pasted at a time is not particularly limited, either, but one sheet is enough. A recommended pasting duration at a time is 6 to 24 hours.

### EXAMPLES

Examples are shown below, and the present invention will be described more specifically. It should be noted that the present invention is not limited to these Examples, but various changes are possible within a range not departing from the technical idea of the present invention.

### Manufacture Method of Patch-Type Supplement Sheet

After the supplement component was dissolved or dispersed in water or an organic solvent in accordance with the component, it was added to an adhesive solution and stirred well and homogenized so as to obtain a coating solution. In Examples 1 to 4, the coating solution was applied to a PET film which has been subjected to releasing treatment by a table coating machine so that the thickness after being dried is 40 μm and was dried at 80° C. for 30 minutes in a gear oven so as to volatilize the solvent. The obtained sheet was cut into a regular square of 4 cm×4 cm so as to manufacture the patch-type supplement sheet.

### Example 1: Manufacture of Patch-Type Supplement Sheet (Supplement 1)

The patch-type supplement sheet (supplement 1) was manufactured by using materials shown in Table 1. More specifically, the materials in a group D were stirred and mixed so as to obtain a uniform solution (1). The mixed materials in a group A were added to (1) and stirred and mixed (2). The materials in a group B and a group C were heated to 50 to 60° C., respectively, and uniformly mixed/dissolved were added to (2), stirred and homogenized so as to obtain the coating solution.

TABLE 1

| | | | Material in | |
|---|---|---|---|---|
| | | | coating liquid | Material/sheet |
| Group | Name of material | Supplier or manufacturer | (g) | (mg/one sheet) |
| A | Panax ginseng root essence | INA TRADIING CO., LTD | 5 | 2.5 |
| | American ginseng root essence | | 5 | 2.5 |
| | Eleutherococcus senticosus root | | 5 | 2.5 |
| | essence | | | |
| | Ophiocordyceps sinensis | MARUZEN PHARMACEUTICALS | 10 | 5 |
| | | CO., LTD. | | |
| B | Taurine | | 4 | 4 |
| | Arginine | Nippon Bulk Yakuhin | 1 | 1 |
| | | Co., Ltd. | | |
| | PG | | 1 | 1 |
| | Purified water | | 60 | |
| C | Pycnogenol | Nippon Bulk Yakuhin | 0.5 | 0.5 |
| | | Co., Ltd. | | |
| | Ethanol | | 50 | |
| D | Ethyl acetate | WAKENYAKU CO., LTD. | 100 | |
| | HIPAS-10(Hydrophilic acrylic | CosMED Pharmaceutical | 300 | 105 |
| | adhesive solution) | Co., Ltd. | | |
| | Isopropyl myristate | Nippon Bulk Yakuhin | 30 | 30 |
| | | Co., Ltd. | | |

### Example 2: Manufacture of Patch-Type Supplement Sheet (Supplement 2)

The patch-type supplement sheet (supplement 2) was manufactured by using materials shown in Table 2. More specifically, the materials in the group A and the group B were heated to 50 to 60° C., respectively, and dissolved. The materials in the group D were stirred, mixed, and uniformly dissolved. The materials in the three groups in total, that is, the dissolved materials in the group A and the group B and the material in the group C were stirred, mixed and uniformly mixed (1). The dissolved material in the group D was added to (1), well stirred to be homogenized so as to obtain the coating solution.

TABLE 2

| | | | Material in | |
|---|---|---|---|---|
| | | | coating liquid | Material/sheet |
| Group | Name of material | Manufacturer | (g) | (mg/one sheet) |
| A | Curcumin | Sabinsa Japan Corporation | 0.5 | 0.5 |
| | Ethanol | WAKENYAKU CO., LTD. | 70 | |
| | Silybum marianum extract powder- | BGG Japan Co., Ltd. | 0.5 | 0.5 |
| | BGG-J | | | |
| B | Lactic fermented barley liquid | OSAKA SASAKI CHEMICAL | 2.5 | 2.5 |
| | GABA 90% | Co., Ltd. | | |
| | Ornithine hydrochloride | | 0.5 | 0.5 |
| | Black ginger essence | | 0.5 | 0.5 |
| | Liver HI | | 1 | 1 |
| | Purified water | | 50 | |
| C | Curcuma domestica | MARUZEN | 5 | 2.5 |
| | essence | PHARMACEUTICALS | | |
| | | CO., LTD. | | |
| D | HIPAS-10(Hydrophilic acrylic based | CosMED Pharmaceutical | 300 | 105 |
| | adhesive solution) | Co., Ltd. | | |
| | Acetone | | 50 | |
| | IPM (Isopropyl myristate) | Nippon Bulk Yakuhin | 30 | 30 |
| | | Co., Ltd. | | |

### Example 3: Manufacture of Patch-Type Supplement Sheet (Supplement 3)

The patch-type supplement sheet (supplement 3) was manufactured by using materials shown in Table 3. More specifically, the materials in the group A were mixed/stirred at 50 to 60° C. (1). After the materials in the group B were made into a solution at 50 to 60° C., it was added to (1) so as to obtain a uniform solution (2). After the materials in the group C were stirred and mixed so as to obtain the uniform solution, (2) was added to that and stirred and homogenized so as to obtain the coating solution.

TABLE 3

| | | | Material in | |
|---|---|---|---|---|
| | | | coating liquid | Material/sheet |
| Group | Name of material | Manufacturer | (g) | (mg/one sheet) |
| A | Vitamin premix RD-2001 | DSM | 15 | 15 |
| | Purified water | | 60 | |
| B | Nicotinic acid amide | OSAKA SASAKI CHEMICAL | 2.5 | 2.5 |
| | | Co., Ltd. | | |
| | Curcumin | Sabinsa Japan Corporation | 0.5 | 0.5 |
| | Ethanol | WAKENYAKU CO., LTD. | 50 | |
| C | Ethyl acetate | WAKENYAKU CO., LTD. | 100 | |
| | IPM (Isopropyl myristate) | Nippon Bulk Yakuhin | 30 | 30 |
| | | Co., Ltd. | | |
| | HIPAS-10(Hydrophilic acrylic | CosMED Pharmaceutical | 300 | 105 |
| | based adhesive) | Co., Ltd. | | |

### Example 4: Manufacture of Patch-Type Supplement Sheet (Supplement 4)

The patch-type supplement sheet (supplement 4) was manufactured by using materials shown in Table 4. More specifically, the materials in the group A were added to the materials in the group D and uniformly mixed (1). The materials in the group B and the group C were heated to 50 to 60° C., respectively, so as to obtain a solution and then, added to (1), well stirred and homogenized so as to obtain the coating solution.

TABLE 4

| | | | Material in | |
|---|---|---|---|---|
| | | | coating liquid | Material/sheet |
| Group | Name of material | Manufacturer | (g) | (mg/one sheet) |
| A | Vitamin A palmitate | RIKEN VITAMIN CO., LTD. | 5 | 5 |
| | Vitamin E acetate | RIKEN VITAMIN CO., LTD. | 30 | 30 |
| | VC-IP | OSAKA SASAKI | 10 | 10 |
| | Tocoretinoate-10 | CHEMICAL Co., Ltd. | 0.5 | 0.5 |
| | β-carotene | | 0.5 | 0.5 |
| | Ethanol | | 50 | |
| B | CoQ-10 | KANEKA CORPORATION | 0.04 | 0.04 |
| | α-lipoic acid | Oryza Oil & Fat | 0.015 | 0.015 |
| | | Chemical Co., Ltd. | | |
| | IPM(Isopropyl | Nippon Bulk Yakuhin Co., | 30 | 30 |
| | myristate) | Ltd. | | |
| C | FujiflavoneP-10 | Nippon Bulk Yakuhin Co., | 0.5 | 0.5 |
| | | Ltd. | | |
| | Pomegranate rind | | 5 | 2.5 |
| | essence | | | |
| | Purified water | | 30 | |
| D | Ethyl acetate | WAKENYAKU CO., LTD. | 50 | |
| | HIPAS-10(Hydrophilic | CosMED Pharmaceutical | 300 | 105 |
| | acrylic based adhesive) | Co., Ltd. | | |
| | Polyisocyanate | Nippon Polyurethane | 0.1 | |
| | | Industry Co., Ltd. | | |

### Example 5: Manufacture of Patch-Type Supplement Sheet (Supplement 5)

The patch-type supplement sheet (supplement 5) was manufactured by using materials shown in Table 5. More specifically, the group D was dissolved at 150° C., the materials in the group A were added to the group D cooled to 100° C. and mixed uniformly (1). The manufacture was similar to Examples 1 to 4 except that the materials in the group B and the group C were heated to 50 to 60° C., dissolved and added to (1) and then, well stirred and homogenized so as to obtain the coating solution, and the coating was performed at 110° C.

TABLE 5

| | | | Material in | |
|---|---|---|---|---|
| | | | coating liquid | Material/sheet |
| Group | Name of material | Manufacturer | (g) | (mg/one sheet) |
| A | Vitamin A palmitate | RIKEN VITAMIN CO., LTD. | 5 | 5 |
| | Vitamin E acetate | RIKEN VITAMIN CO., LTD. | 30 | 30 |
| | VC-IP | OSAKA SASAKI | 10 | 10 |
| | Tocoretinoate-10 | CHEMICAL Co., Ltd. | 0.5 | 0.5 |
| | β-carotene | | 0.5 | 0.5 |
| B | CoQ-10 | KANEKA CORPORATION | 0.01 | 0.01 |
| | α-lipoic acid | Oryza Oil & Fat | 0.01 | 0.01 |
| | | Chemical Co., Ltd. | | |
| | IPM(Isopropyl myristate) | Nippon Bulk Yakuhin | 10 | 10 |
| | | Co., Ltd. | | |
| C | FujiflavoneP-10 | Nippon Bulk Yakuhin | 0.1 | 0.1 |
| | | Co., Ltd. | | |
| | Pomegranate rind | WAKENYAKU | 5 | 2.5 |
| | essence | CO., LTD. | | |
| | BG | | 5 | 5 |
| D | SIS | Shima Trading Co., | 50 | 50 |
| | | Ltd. | | |
| | Liquid paraffin | NACALAI TESQUE, | 100 | 100 |
| | | INC. | | |

### Comparative Example 1: Manufacture of Supplement Patch (Supplement 6)

The supplement patch (supplement 6) was manufactured by using the materials shown in Table 6. More specifically, the group D and the group C were well mixed and dissolved (1). The materials in the group A and the group B were well mixed and uniformly mixed (2). After (2) was added to (1), it was well stirred and homogenized so as to obtain the coating solution and spread directly on a white unwoven cloth so that the thickens is 100 μm in the coating and then, dried at a room temperature.

TABLE 6

| | | | Material in | |
|---|---|---|---|---|
| | | | coating | Material/sheet |
| Group | Name of material | Manufacturer | liquid (g) | (mg/one sheet) |
| A | Vitamin A palmitate | RIKEN VITAMIN CO., LTD. | 0.1 | 0.1 |
| | Vitamin E acetate | RIKEN VITAMIN CO., LTD. | 1 | 1 |
| | VC-IP | OSAKA SASAKI | 0.5 | 0.5 |
| | Tocoretinoate-10 | CHEMICAL Co., Ltd. | 0.5 | 0.5 |
| | β-carotene | | 0.5 | 0.5 |
| | Tween 80 | | 0.2 | 0.2 |
| B | CoQ-10 | KANEKA CORPORATION | 0.01 | 0.01 |
| | α-lipoic acid | Oryza Oil & Fat | 0.01 | 0.01 |
| | | Chemical Co., Ltd. | | |
| | Ethanol | Nippon Bulk Yakuhin | 5 | 5 |
| | | Co., Ltd. | | |
| C | FujiflavoneP-10 | Nippon Bulk Yakuhin | 0.5 | 0.5 |
| | | Co., Ltd. | | |
| | Pomegranate rind | WAKENYAKU | 5 | 2.5 |
| | essence | CO., LTD. | | |
| | Water | | 10 | 10 |
| D | Glycerin | OSAKA SASAKI | 25 | 25 |
| | | CHEMICAL Co., Ltd. | | |
| | Carmellose sodium | Nippon Bulk Yakuhin | 8.5 | 8.5 |
| | (CMC-Na) | Co., Ltd. | | |
| | Tartaric acid | WAKENYAKU CO., LTD. | 0.5 | 0.2 |
| | Aluminum hydroxide gel | WAKENYAKU CO., LTD. | 0.2 | 0.2 |
| | Water | | 50 | 50 |

### Evaluation Test

The sheets (regular square of 4 cm×4 cm) in Examples 1 to 5 and Comparative Example 1 obtained as above were pasted to upper arm parts of 14 volunteers and released after 6 hours, and redness on the skin and remaining glue were observed.

TABLE 7

| | After pasting | | Evaluation | | |
|---|---|---|---|---|---|
| | | Redness | Re- | Appli- | Water vapor |
| | Peeling- | on | maining | cation | permeability |
| | off | skin | glue | feeling | (g/m² × 24 hrs) |
| Example 1 | 1 | 1 | 1 | 1 | 63 |
| Example 2 | 1 | 1 | 1 | 1 | 70 |
| Example 3 | 1 | 1 | 1 | 1 | 65 |
| Example 4 | 1 | 1 | 1 | 1 | 67 |
| Example 5 | 1 | 1 | 1 | 1 | 60 |
| Comparative | 3* | | | | 2000 or more |
| example 1 | | | | | |

*Evaluation could not be made for the other evaluation items because the sheet had no adhesion property and was peeled off the skin immediately.

### Redness on the Skin

1. No redness
2. Slight redness
3. Clear redness

### Remaining Glue

1. No remaining glue
2. Slight remaining glue
3. Large remaining glue

### Peeling-Off

1. No peeling-off
2. Slight peeling-off
3. Entire peeling-off

### Application Feeling

1. Favorable
2. Slight itching, discomfort
3. Intense itching, discomfort

### Example 6

A sheet with the thickness of 50 μm containing 5.1 mass % of α-lipoic acid in an HIPAS-10 adhesive was manufactured, and transdermal permeability of α-lipoic acid was evaluated.

An automated sampling system TransView C12 for in vitro transdermal permeability test by CosMED Pharmaceutical Co., Ltd. was used for the in vitro transdermal permeability test. The skin (φ22 to 25 mm)* on which a sample patch (φ¹³ mm) was pasted was mounted on a Franz-type permeability diffusion cell and filled with a receptor liquid (ethanol:phosphate buffer=10:90 (% v/v), kept at 32° C. and sampled (500 μL was sampled) after 24 hours from start of the test. The obtained receptor solution was analyzed by HPLC, and a penetration amount per unit area was quantitatively determined.

*Specification of the skin extracted from a human was as follows: Dermatome (made of keratin, epiderm, and a part of derma) used.

The obtained penetration amount for 24 hours was 450 μg/cm².

1. A patch-type supplement composed of a support, a component-containing layer, and a release liner, wherein the component-containing layer contains a supplement component and an adhesive.

2. The patch-type supplement according to claim 1, wherein a water vapor permeability of the support is not larger than 1000 g/m²×24 hours.

3. The patch-type supplement according to claim 1, wherein the supplement component is made of an aqueous component and an organic component.

4. The patch-type supplement according to claim 1, wherein the supplement component is made of the aqueous component and the organic component, and two kinds or more of each of the components are contained.

5. The patch-type supplement according to claim 1, wherein at least one kind of the supplement component exists in the component-containing layer in a dissolved state, and the other at least one kind exists in the component-containing layer in a dispersed state.

6. The patch-type supplement according to claim 1, wherein at least one kind of the supplement component is dispersed in the component-containing layer in a powder state.

7. The patch-type supplement according to claim 1, which is a matrix-type supplement sheet.

8. The patch-type supplement according to claim 1, wherein the component-containing layer further contains at least one kind of a dissolving agent or a surfactant.

9. The patch-type supplement according to claim 1, wherein the total of the supplement components is 5 mass parts or more to 100 mass parts of the component-containing layer.

10. The patch-type supplement according to claim 9, wherein the total of the aqueous component or the total of the organic component of the supplement component is 1 mass part or more to the 100 mass parts of the component-containing layer.

11. The patch-type supplement according to claim 1, wherein the adhesive is an acrylate-based, styrene-isoprene-styrene (SIS)-based, polyisobutylene (PIM-based, or silicone-based adhesive.

12. The patch-type supplement according to claim 1, wherein the adhesive is an acrylate-based adhesive, and the adhesive is crosslinked.

13. The patch-type supplement according to claim 1, wherein the adhesive is a hydrophilic acrylate-based adhesive.

14. The patch-type supplement according to claim 1, wherein the component-containing layer is a lamination of the supplement component-containing layer and a supplement component-free layer.
